(19) United States
(12) Patent Application Publication
(10) Pub. No.: US 2020/0338166 A1
(43) Pub. Date: Oct. 29, 2020

(54) ANTI-TUMOR DRUG COMPOSITION AND POLYNUCLEOTIDE COMPOSITION

(71) Applicant: BEIJING KENUOKEFU BIOTECHNOLOGY CO., LTD., Beijing (CN)

(72) Inventor: Jinyu ZHANG, Beijing (CN)

(73) Assignee: BEIJING KENUOKEFU BIOTECHNOLOGY CO., LTD., Beijing (CN)

(21) Appl. No.: 16/921,969

(22) Filed: Jul. 7, 2020

Related U.S. Application Data
(63) Continuation of application No. PCT/CN2019/070994, filed on Jan. 9, 2019.

(30) Foreign Application Priority Data
Feb. 1, 2018 (CN) 201810104146.9

(51) Int. Cl. A61K 38/20; A61K 38/19; A61P 35/00

(52) U.S. Cl. CPC A61K 38/208; A61P 35/00; A61K 38/193; A61K 38/2013

(57) ABSTRACT

An anti-tumor drug composition, including IL 12 proteins, GMCSF proteins, and IL 2 proteins.

### CROSS REFERENCE TO RELATED APPLICATIONS

The present application is the continued application of PCT application No. PCT/CN2019/070994 filed on Jan. 9, 2019, which claims priority to Chinese Application No. 201810104146.9 filed on Feb. 1, 2018, all of which are incorporated herein by reference in its entirety for all purposes.

### TECHNICAL FIELD

This application relates to a field of an anti-tumor drug, and more particularly to an anti-tumor protein composition.

### BACKGROUND

Tumor is a neogrowth formed by clonal hyperplasia when a cell of local tissue can not regulate its normal growth at the genetic level due to various carcinogenic factors, and tumor is also called neoplasm since most of the neogrowths are protruded as a space-occupying lump.

In recent years, there are many new ways developed for tumor therapy. Radiotherapy, chemotherapy, surgical therapy, and immunotherapy are current commonly-used therapeutic means. However, problems such as serious adverse reactions during radiotherapy and chemotherapy, high risk caused by surgical therapy, and poor effect of the immunotherapy against solid tumors still exist.

### BRIEF SUMMARY

In light of the above problems, the present application provides an anti-tumor protein composition having the following advantages: 1. minor adverse reactions; 2. good inhibitory effect against various solid tumors which can reduce or even eliminate the tumor; 3. good inhibitory effect against the metastasis of malignant tumors; and 4. easy implementation by existing technologies.

In a first aspect of the present application, an anti-tumor drug composition is provided, including IL12 proteins, GMCSF proteins, and IL2 proteins.

By the above-mentioned technical solution, the symptoms in patients suffering from a plurality of solid tumors may be well controlled, and some conditions may even be completely relieved. It has little stimulation to a patient, and with minor adverse reactions, which greatly improves the life quality of the patient.

Preferably, in the composition, a mass ratio of the IL12 protein: the GMCSF protein: the IL2 protein is in ranges of 0.1-10:0.1-10:0.1-10.

Preferably, in the composition, the mass ratio of the IL12 protein: the GMCSF protein: the IL2 protein is in ranges of 0.6-6:0.6-6:0.6-6.

Preferably, in the composition, the mass ratio of the IL12 protein: the GMCSF protein: the IL2 protein is 1:6:2.

In a second aspect of the present application, a polynucleotide composition is provided, and including one selected from the group consisting of:

(a) a polynucleotide composition coding the IL12 protein, the GMCSF protein, and the IL2 protein according to the first aspect; and
(b) a polynucleotide composition complementary to the polynucleotide composition in (a).

In a third aspect of the present application: a recombinant vector composition is provided, which is constructed from each polynucleotide of the polynucleotide composition according to the second aspect with a plasmid, a virus, and an expressing vector, respectively.

In a fourth aspect of the present application: a genetically engineered host cell composition is provided, wherein a host cell of the host cell composition includes one of the following: (a) a host cell transformed or transduced from the recombinant vector according to the third aspect; and (b) a host cell transformed or transduced from the polynucleotide composition according to the second aspect.

In a fifth aspect of the present application: an anti-tumor drug is provided, including the anti-tumor drug composition according to the first aspect, or the polynucleotide composition according to the second aspect, or the recombinant vector composition according to the third aspect, or the host cell composition according to the fourth aspect, and a pharmaceutically acceptable carrier or buffer or additive or excipient or adjuvant.

In a sixth aspect of the present application: an application of the anti-tumor drug composition according to the first aspect, or the polynucleotide composition according to the second aspect, or the recombinant vector composition according to the third aspect, or the host cell composition according to the fourth aspect, or the anti-tumor drug according to the fifth aspect in a field of a drug treating tumors in a mammal.

Preferably, the mammal includes Primates other than human beings, Diprotodontia, Carnivora, Perissodactyla, Artiodactyla, Rodentia, and Lagomorpha.

Preferably, the tumor includes melanoma, kidney cancer, prostate cancer, breast cancer, colon cancer, lung cancer, liver cancer, bone cancer, pancreatic cancer, skin cancer, head or neck cancer, oral cancer, nasopharyngeal cancer, skin or intraocular malignant melanoma, uterine cancer, ovarian cancer, rectal cancer, anal cancer, gastric cancer, testicular cancer, uterine cancer, fallopian tube cancer, endometrial cancer, cervical cancer, vaginal cancer, vulvar cancer, Hodgkin's disease, non-Hodgkin's lymphoma, esophageal cancer, small intestine cancer, thyroid cancer, parathyroid cancer, adrenal cancer, soft tissue sarcoma, urethral cancer, penile cancer, acute myeloid leukemia, chronic myeloid leukemia, acute lymphoblastic leukemia, chronic lymphocytes leukemia, lymphocytic lymphoma, bladder cancer, renal or ureteral cancer, renal pelvis cancer, central nervous system neoplasm, primary central nervous system lymphoma, tumor angiogenesis, spinal cord tumor, brain stem glioma, pituitary adenoma, Kaposi's sarcoma, epidermoid carcinoma, squamous cell carcinoma, and T cell lymphoma.

In a seventh aspect of the present application: the anti-tumor drug composition according to the first aspect, or the polynucleotide composition according to the second aspect, or the recombinant vector composition according to the third aspect, or the host cell composition according to the fourth aspect, or the anti-tumor drug according to the fifth aspect is injected into an affected area of animal or introduced into an affected area of a tumor by intravenous injection through a targeted carrier.

In summary, the embodiments of the present application have the following beneficial effects:

1. minor adverse reactions, which is commonly a transitory fever;

2. good inhibitory effect against various solid tumors which can reduce or even eliminate the tumor;

3. good inhibitory effect against the metastasis of malignant tumors; and

4. greatly improved quality of life of a patient.

### BRIEF DESCRIPTION OF DRAWINGS

FIG. 1 is a diagram showing the treatment effect on an experimental animal in Example 5.

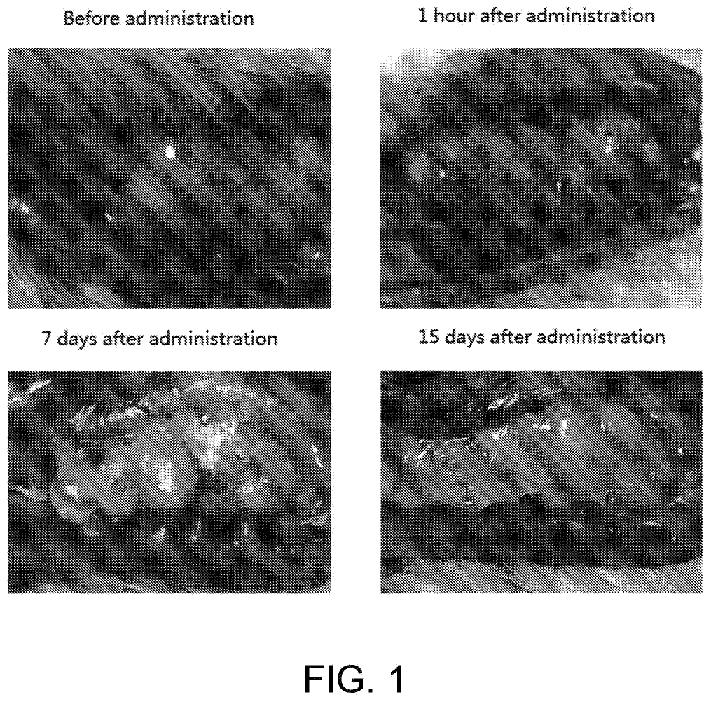

FIG. 2 is a diagram showing the treatment effect on an experimental animal in Example 6.

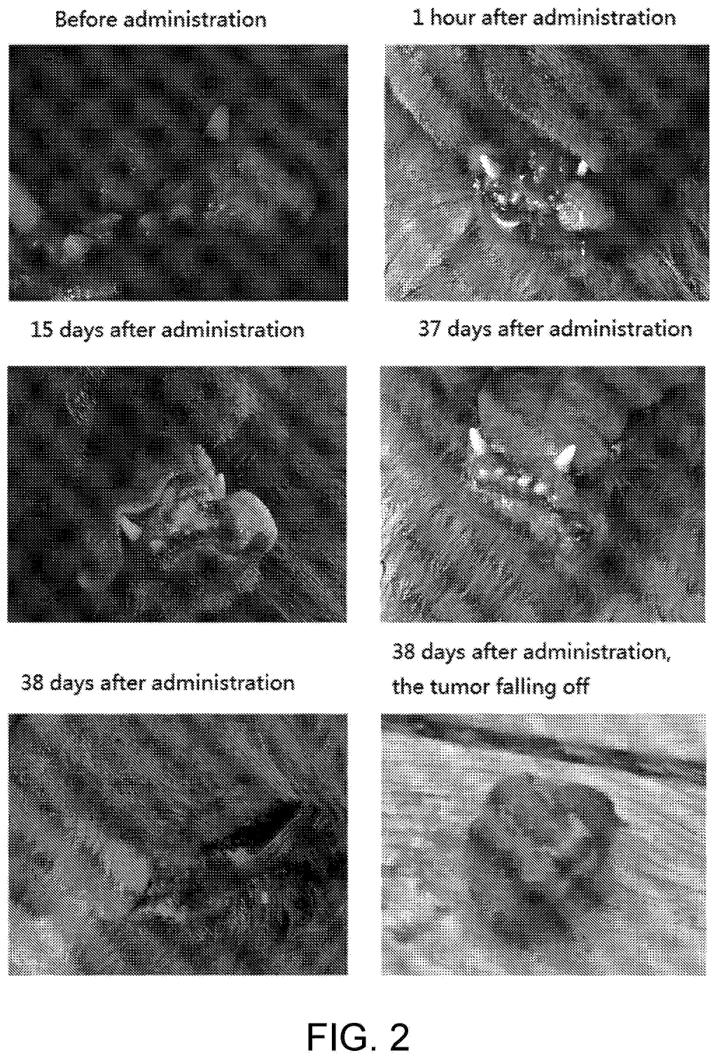

FIG. 3 shows sequences of IL12 protein, GMCSF protein, and IL2 protein of dog.

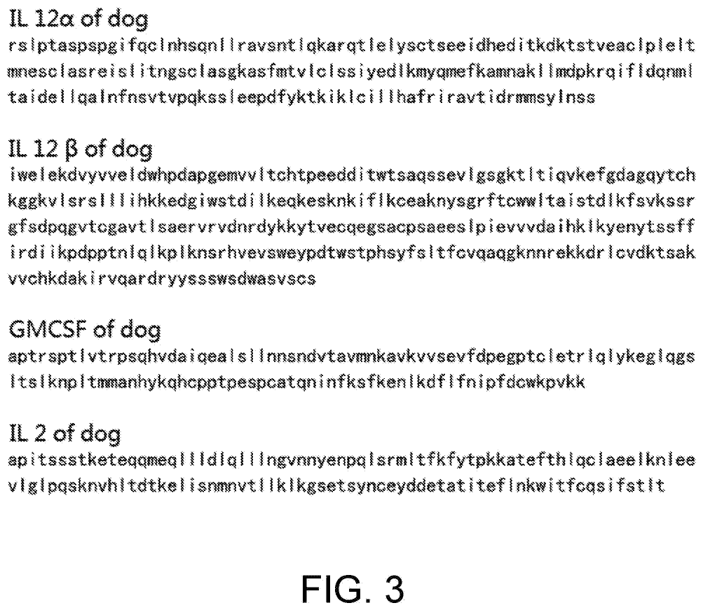

FIG. 4 shows sequences of IL12 protein, GMCSF protein, and IL2 protein of cat, which are consistent with those provided in the nucleotide sequence list attached hereto.

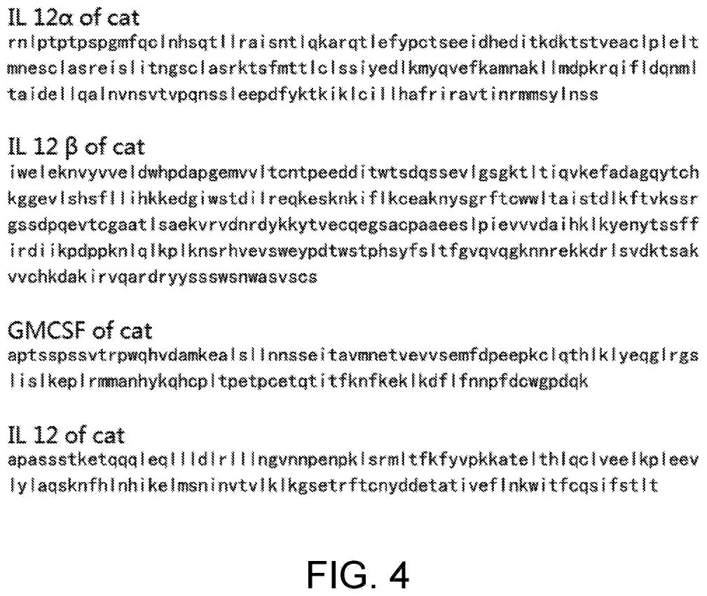

### DETAILED DESCRIPTION

This application will be further explained in detail as follow.

Reagents: DMEM medium, 1640 medium, fetal bovine serum purchased from lifetechnologies company; CDM4HEK293 serum free medium purchased from Thermo company; cell culture flask and culture plate purchased from Corning company; Puromycin purchased from Chemicon company; restriction endonuclease purchased from Takara and NEB company; ligase purchased from NEB company; DNA polymerase purchased from Takara company; Plasmid Extraction Kit and Gel Extraction Kit purchased from OmegaBiotech company; primer synthesized by Sangon Biotech (Shanghai) Co., Ltd; and gene synthesis and sequencing approached by lifetechnologies company. IL12 ELISA kit and IL2 ELISA kit purchased from Thermo company; GMCSF ELISA kit purchased from Sigma company; and chitosan (Protosan G 213) purchased from NovaMatrix company. Recombinant dogs IL12, GMCSF, IL2 protein, recombinant cats IL12, GMCSF, IL2 protein, purchased from NovusBiologicals company.

### Example 1: Construction of Cells Expressing IL12

The coding region of dog IL12 gene including two subunits IL12a (Genbank No: NM_001003293) and IL12β (Genbank No: NM_001003292) was synthesized and these two subunits were ligated by T2A sequence. Two ends of the synthesized gene include restriction sites BamHI and XhoI, respectively. Then the synthesized gene was cleaved by BamHI and XhoI via the following system: 5 μg IL12 plasmid, 4 μL digestion buffer, 1 μL BamHI, 1 μL XhoI, a total volume of 40 μL obtained by adding water, and 12 hours of standing at 37° C. The Eppendorf was taken out, and 4.4 μL 10× loading buffer was added to the eppendorf. An electrophoresis was carried out by using 1% agarose gel, after which fragments of IL12 gene were recovered for use.

Expression vector pLentis-CMV-MCS-IRES-PURO was cleaved via the following system: 2 μg plasmid, 3 μL digestion buffer, 1 μL BamHI, 1 μL XhoI, a total volume of 30 μL obtained by adding water, and 12 hours of standing at 37° C. Eppendorf was taken out, and 3.3 μL 10× loading buffer was added to the eppendorf. An electrophoresis was carried out by using 1% agarose gel, after which fragments of the vector were recovered for use.

PLentis-CMV-MCS-IRES-PURO and IL12 were ligated via the following system: 2 μL pLentis-CMV-MCS-IRES-PURO, 2 μL IL12, 1 μL ligase buffer, 0.5 μL T4 DNA ligase, and 4.5 μL water. The ligation was performed at room temperature for 4 hours. Then competent cells from Escherichia coli were transformed into the ligated system. The next day, bacterial colonies were picked up from the transformed plate, placed in LB medium and cultured in a shaker at a temperature of 37° C. overnight. Plasmid was extracted from the cultured bacterium by using the Plasmid Extraction Kit, and fragments were cleaved and identified whether they had been successfully ligated into vectors. Desired vectors were sequenced for examination, and it was confirmed that the expression vector pLentis-CMV-IL12-PGK-PURO had been successfully constructed.

Virus for regulating the vector was prepared by the following method: 1. Cultured 293FT cells were digested, counted, and placed into wells of a 10-cm culture plate by 3×10⁶ cells/well, with the volume of the culture solution being 10 ml. 2. At the next night, the state of cells was observed, and desired cells were transfected. Chloroquine was added to the plate until a final concentration of 25 μm was reached. Sterile water and a plasmid (pMD2.G 5 μg+pSPAX2 15 μg+pLentis-CMV-IL12-PGK-PURO 20 μg) were added to a test tube to reach a total volume of 1045 μL. Then 155 μL of 2M CaCl₂ was added to the test tube and mixed evenly. Finally 1200 μL 2×HBS was added to the test tube under shaking. The mixture was quickly added into the wells of the plate, and mixed evenly under a gentle shaking. 3. On the morning of the third day, the state of cells was observed, and the medium was replaced by 10 ml fresh DMEM medium. 4. On the morning of the fifth day, the state of cells was observed, and supernatant in the plate was collected, filtered through a 0.45 μm filter, and centrifuged for 2 hours in a high-speed centrifuge tube at 50000 g. The supernatant was carefully removed. The precipitate was dried by an absorbent paper, resuspended in 500 μL HBSS for 2 hours, sub-packed in several tubes and preserved at −70° C.

Virus was used to transfect 293 cells by the following method: cultured 293 cells were digested, and inoculated into the wells of a 6-well plate by 10⁵ cells/well, in which the volume of the culture solution was 1 ml. After 24 hours, 10 μL virus for regulating the vector was added, and cells were cultured in the incubator for another 24 hours. Then, the supernatant was removed, and fresh medium was added. After the surface was fully covered with cells, cells were transferred to a flask. Puromycin at a suitable concentration was added to the flask, and culturing was continued, during which the medium was replaced every two days and the concentration of puromycin was kept at 3 μg/ml. After one week of screening, viable cells were acquired, that is, cells stably expressing regulation protein, which were named 293(1E12).

### Example 2: Construction of Cells Expressing GMCSF

The coding region of dog GMCSF gene (Genbank No: NM_001003245) was synthesized. Two ends of the synthesized gene include restriction sites BamHI and XhoI, respectively. Then the synthesized gene was cleaved by BamHI and XhoI via the following system: 5 μg GMCSF plasmid, 4 μL digestion buffer, 1 μL BamHI, 1 μL XhoI, a total volume of 40 μL obtained by adding water, and 12 hours of standing at 37° C. Eppendorf was taken out, and 4.4 μL 10× loading buffer was added to the eppendorf. An electrophoresis was carried out by 1% using agarose gel, after which the fragments of GMCSF gene were recovered for use.

Expression vector pLentis-CMV-MCS-IRES-PURO was cleaved via the following system: 2 μg plasmid, 3 μL digestion buffer, 1 μL BamHI, 1 μL XhoI, a total volume of 30 μL obtained by adding water, and 12 hours of standing at 37° C. Eppendorf was taken out, and 3.3 μL 10× loading buffer was added to the eppendorf. An electrophoresis was carried out by using 1% agarose gel, and fragments of the vector were recovered for use.

PLentis-CMV-MCS-IRES-PURO and GMCSF were ligated via the following system: 2 μL pLentis-CMV-MCS-IRES-PURO, 2 μL GMCSF, 1 μL ligase buffer, 0.5 μL T4 DNA ligase, and 4.5 μL water. The ligation was performed at room temperature for 4 hours. Then competent cells from Escherichia coli were transformed into the ligated system. The next day, bacterial colonies were picked up from the transformed plate, placed in LB medium and cultured in a shaker at a temperature of 37° C. overnight. Plasmid was extracted from the cultured bacterium by using the Plasmid Extraction Kit, and fragments were cleaved and identified whether they had been successfully ligated into the vectors. Desired vectors were sequenced for examination, and it was confirmed that the expression vector pLentis-CMV-GMCSF-PGK-PURO had been successfully constructed.

Virus for regulating the vector was prepared by the following method: 1. cultured 293FT cells were digested, counted, placed into the wells of a 10-cm culture plate by 3×10⁶ cells/well, in which the volume of the culture solution was 10 ml. 2. At the next night, the state of cells was observed, and desired cells were transfected. Chloroquine was added to the plate until a final concentration of 25 μm was reached. Sterile water and a plasmid (pMD2.G 5 μg+pSPAX2 15 μg+pLentis-CMV-IL12-PGK-PURO 20 μg) were added to a test tube to reach a total volume of 1045 μL. Then 155 μL of 2M CaCl₂ was added to the test tube and mixed evenly. Finally, 1200 μL 2×HBS was added to the test tube under shaking. The mixture was quickly added into the wells of the plate, and mixed evenly under a gentle shaking. 3. On the morning of the third day, the state of cells was observed, and the medium was replaced by 10 ml fresh DMEM medium. 4. On the morning of the fifth day, the state of cells was observed, and supernatant in the plate was collected, filtered through 0.45 μm filter, and centrifuged in a high-speed centrifuge tube at 50000 g for 2 hours. The supernatant was carefully removed, and the precipitate was dried by an absorbent paper, resuspended in 500 μL HBSS for 2 hours, sub-packed into several tubes and preserved at −70° C.

Virus was used to transfect 293 cells by the following method: cultured 293 cells were digested, and inoculated into a 6-well plate by 10⁵ cells/well, in which the volume of the culture solution was 1 ml. After 24 hours, 10 μL virus for regulating the vector was added, and cells were cultured in the incubator for another 24 hours. Then, the supernatant was removed, and fresh medium was added. After the surface was fully covered with cells, cells were transferred into a flask. Puromycin at a suitable concentration was added, and culturing was continued, during which medium was replaced every two days, and the concentration of puromycin was kept at 3 μg/ml. After one week of screening, viable cells were acquired, that is, cells stably expressing regulation protein, which were named 293(GMCSF).

### Example 3: Construction of Cells Expressing IL2

The coding region of dog IL2 gene (Genbank No: NM_001003305) was synthesized. Two ends of the synthesized gene include restriction sites BamHI and XhoI, respectively. Then the synthesized gene was cleaved by BamHI and XhoI via the following system: 5 μg IL2 plasmid, 4 μL digestion buffer, 1 μL BamHI, 1 μL XhoI, a total volume of 40 μL obtained by adding water, and 12 hours of standing at 37° C. Eppendorf was taken out, and 4.4 μL 10× loading buffer was added to the eppendorf. An electrophoresis was carried out by using 1% agarose gel, and fragments of IL2 gene were recovered for use.

Expression vector pLentis-CMV-MCS-IRES-PURO was cleaved via the following system: 2 μg plasmid, 3 μL digestion buffer, 1 μL BamHI, 1 μL XhoI, a total volume of 30 μL obtained by adding water, and 12 hours of standing at 37° C. Eppendorf was taken out, and 3.3 μL 10× loading buffer was added to the eppendorf. An electrophoresis was carried out by using 1% agarose gel, and the fragments of vector were recovered for use.

PLentis-CMV-MCS-IRES-PURO and IL2 were ligated via the following system: 2 μL pLentis-CMV-MCS-IRES-PURO, 2 μL IL2, 1 μL ligase buffer, 0.5 μL T4 DNA ligase, and 4.5 μL water. The ligation was performed at room temperature for 4 hours. Then competent cells from Escherichia coli were transformed into the ligated system. The next day, bacterial colonies were picked up from the transformed plate, placed in LB medium and cultured in a shaker at a temperature of 37° C. overnight. Plasmid was extracted from the cultured bacterium by using the Plasmid Extraction Kit, and fragments were cleaved and identified whether they had been successfully ligated into vectors. Desired vectors were sequenced for examination, and it was confirmed that the expression vector pLentis-CMV-IL2-PGK-PURO had been successfully constructed.

Virus for regulating the vector was prepared by the following method: 1. cultured 293FT cells were digested, counted, and placed into a 10-cm culture plate by 3×10⁶ cells/well, in which the volume of the culture solution was 10 ml. 2. At the next night, the state of cells was observed, and desired cells were transfected. Chloroquine was added to the plate until a final concentration of 25 μm was reached. Sterile water and a plasmid (pMD2.G 5 μg+pSPAX2 15 μg+pLentis-CMV-IL12-PGK-PURO 20 μg) were added to a test tube to reach a total volume of 1045 μL. Then 155 μL of 2M CaCl₂ was added to the test tube and mixed evenly. Finally 1200 μL 2×HBS was added under shaking. The mixture was quickly added into the wells of the plate, and mixed evenly under a gentle shaking. 3. On the morning of the third day, the state of cells was observed, and the medium was replaced by 10 ml fresh DMEM medium. 4. On the morning of the fifth day, the state of cells was observed, and supernatant in the plate was collected, filtered through 0.45 μm filter, and centrifuged in a high-speed centrifuge tube at 50000 g for 2 hours. The supernatant was carefully removed, and the precipitate was dried by an absorbent paper, resuspended in 500 μL HBSS for 2 hours, sub-packed into several tubes and preserved at −70° C.

Virus was used to transfect 293 cells by the following method: cultured 293 cells were digested, and inoculated into a 6-well plate by 10⁵ cells/well, in which the volume of the culture solution was 1 ml. After 24 hours, 10 μL virus for regulating the vector was added, and the culturing was continued in the incubator for another 24 hours. Then, the supernatant was removed, and fresh medium was added. After the surface was fully covered with cells, cells were transferred into a flask. Puromycin at a suitable concentration was added, and culturing was continued, during which the medium was replaced every two days, and the concentration of puromycin was kept at 3 μg/ml. After one week of screening, viable cells were acquired, that is, cells stably expressing regulation protein, which were named 293(IL2).

### Example 4: Preparation of Protein Solution

Cultured cells 293(IL12), 293(GMCSF), and 293(IL2) were transferred to a 15 cm culturing dish, respectively, in which the medium was complete medium having volume of 25 ml. When the density of cells reached 90% or above, the complete medium was replaced by 25 ml CDM4HEK293 serum free medium and culturing was continued for another 96 hours. The supernatant was collected, centrifuged at 1000 rpm for 10 min, and filtered through 0.22 μm filter. Filtered solution was concentrated by an Amicon μLtra-15 ultrafiltration tube to one twentieth of the original volume. The concentration of target proteins in the concentrated solution was detected by ELISA kit. The concentration of IL12 was 100 ng/μL, the concentration of GMCSF was 600 ng/μL, and the concentration of IL2 was 200 ng/μL.

### Example 5: Therapy 1 of Tumor in Dogs

Male hybrid dog, 9 years old, 2 sarcomas outside the gingiva on the left upper jaw, the size of the anterior tumor was 25 mm×15 mm, and the size of the posterior tumor was 30 mm×20 mm. As shown in Table 1, after the first administration, the anterior tumor was eliminated, and the area of the posterior tumor was reduced by 70%. Transient fever appeared 8 hours after the administration, body temperature increased by 1° C. and returned to normal after 3 hours, and then all signs became normal.

A 3% sterile chitosan solution was prepared in advance for use. The dosage of drugs to be injected was prepared according to the area of the tumor, and the total dosage was 1 μL/1 mm² tumor. For a tumor with a size of about 900 mm², the injection dosage was 900 μL, including ⅙ volume of IL12 solution, ⅙ volume of GMCSF solution, ⅙ volume of IL2 solution, and ½ volume of 3% chitosan solution. 150 μL IL12 solution, 150 μL GMCSF solution, and 150 μL IL2 solution were mixed evenly, then 450 μL 3% chitosan solution was added, and mixed evenly under slow blowing to avoid bubbles. After the dog was anesthetized, prepared drug solution was injected into the tumor slowly, and the physiological state of the dog was monitored.

TABLE 1
table of size of tumor after the drug was injected in example 5

| Time(day) | 0 | 7 | 14 | 21 | 28 | 35 |
|---|---|---|---|---|---|---|
| Anterior | 25 × 15 | 10 × 6 | 0 | 0 | 0 | 0 |
| tumor(mm) | | | | | | |
| Posterior | 30 × 20 | 25 × 15 | 20 × 15 | 15 × 10 | 15 × 10 | 15 × 10 |
| tumor(mm) | | | | | | |

### Example 6: Therapy 2 of Tumor in Dogs

Canine, chow chow, 11 years old, male, oral melanoma, 2 lesions, 40 mm×30 mm inside the mandibular gingiva, and 30 mm×25 mm outside the mandibular gingiva. As shown in Table 2, after one dose of the drug was injected to melanomas inside and outside the gingiva, respectively, the melanoma inside the gingiva disappeared, and the melanoma outside the gingiva fell off from the root. There was no fever or any adverse reactions observed after administration.

The IL12 solution and IL2 solution were further ultrafiltered and concentrated to 300 ng/μL, and the GMCSF solution was diluted to 300 ng/μL. Then the drug was injected by IL12:GMCSF:IL2:3% chitosan=1:1:1:3, and the total injection volume was 1 μL/mm² tumor. For a tumor with a size of about 900 mm², the injection dosage was 900 μL, including 45 μg IL12, 45 μg GMCSF, and 45 μg IL2. After the dog was anesthetized, prepared drug solution was injected into the tumor slowly, and the physiological state of the dog was monitored.

TABLE 2
table of size of the tumor after the drug was injected in Example 6

| Time(day) | 0 | 7 | 14 | 21 | 28 | 35 | 42 |
|---|---|---|---|---|---|---|---|
| Inside | 40 × 30 | 40 × 30 | 30 × 25 | 21 × 16 | 12 × 8 | 0 | 0 |
| tumor(mm) | | | | | | | |
| Outside | 30 × 25 | 30 × 25 | 30 × 25 | 30 × 25 | 30 × 25 | 38 × 31 | 0 |
| tumor(mm) | | | | | | | |

### Example 7: Therapy 3 of Tumor in Dogs

Female hybrid dog, 15 years old, breast cancer in the third breast region on the left side, with a tumor size of 55 mm×43 mm. As shown in Table 3, the area of the tumor was reduced by 60% after one intratumoral injection. Fever appeared 8 hours after administration, body temperature increased by up to 2° C., and the body temperature returned to normal after 18 hours. Then all signs became normal with no adverse reactions.

The purchased recombinant dog IL12 was diluted with sterile deionized water to 60 ng/μL, the purchased recombinant dog GMCSF was diluted with sterile deionized water to 600 ng/μL, and the purchased recombinant dog IL2 was diluted with sterile deionized water to 600 ng/μL. Then the drug was injected by IL12:GMCSF:IL2:3% chitosan=1:1:1:3, and the total injection volume was 1 μL/mm² tumor. For a tumor with a size of about 900 mm², the injection dosage was 900 μL, including 9 μg IL12, 90 μg GMCSF, and 90 μg IL2. After the dog was anesthetized, prepared drug solution was injected into the tumor slowly, and the physiological state of the dog was monitored. Changes in the size of the tumor were recorded, and the efficacy was evaluated.

TABLE 3
table of size of tumor after being injected in Example 7

| Time(day) | 0 | 7 | 14 | 21 | 28 | 35 |
|---|---|---|---|---|---|---|
| Size of | 55 × 43 | 61 × 49 | 50 × 42 | 45 × 38 | 39 × 33 | 33 × 28 |
| tumor(mm) | | | | | | |

### Example 8: Therapy 4 of Tumor in Dogs

Female hybrid dog, 10 years old, stromal sarcoma on left front leg, 35 mm×35 mm×40 mm. As shown in Table 4, after one intratumoral injection, the area of the tumor was reduced by 60%, and the volume of the tumor was reduced by 50%. Fever appeared 10 hours after administration, body temperature increased by up to 1.5° C., and body temperature returned to normal after 8 hours. There was no observed adverse reaction.

The purchased recombinant dog IL12 was diluted with sterile deionized water to 300 ng/μL, the purchased recombinant dog GMCSF was diluted with sterile deionized water to 60 ng/μL, and the purchased recombinant dog IL2 was diluted with sterile deionized water to 6000 ng/μL. Then the drug was injected by IL12:GMCSF:IL2:3% chitosan=1:1:1:3, and the total injection volume was 1 μL/mm² tumor. For a tumor with a size of about 900 mm², the injection dosage was 900 μL, including 45 μg IL12, 9 μg GMCSF, and 900 μg IL2. After the dog was anesthetized, prepared drug solution was injected into the tumor slowly, and the physiological state of the dog was monitored. Changes in the size of the tumor were recorded, and the efficacy was evaluated.

TABLE 4
table of size of tumor after the drug injected in Example 8

| Time (day) | 0 | 7 | 14 | 21 | 28 | 35 |
|---|---|---|---|---|---|---|
| Size of | 35 × 35 × 40 | 35 × 35 × 38 | 35 × 35 × 35 | 35 × 35 × 33 | 30 × 30 × 25 | 30 × 30 × 25 |
| tumor(mm) | | | | | | |

### Example 9: Therapy 1 of Tumor in Cat

Female Chinese Dragen-Li, 9 years old, breast cancer, long diameter of 21 mm. As shown in Table 5, after one intratumoral injection, the tumor was eliminated. There was no fever or any adverse reactions after administration.

Purchased recombinant cat IL12, GMCSF, and IL2 were diluted with sterile deionized water to 200 ng/μL. Then the drug was injected by IL12:GMCSF:IL2: 3% chitosan=1:1:1:3, and the total injection volume was 1 μL/mm² tumor. For a tumor with a size of about 900 mm², the injection dosage was 900 μL, including 30 μg IL12, 30 μg GMCSF, and 30 μg IL2. After the cat was anesthetized, prepared drug solution was injected into the tumor slowly, and the physiological state of the cat was monitored. Changes in the size of the tumor were recorded, and the efficacy was evaluated.

TABLE 5
table of size of tumor after the drug was injected in Example 9

| Time(day) | 0 | 5 | 10 | 15 | 20 | 25 |
|---|---|---|---|---|---|---|
| Size of | 21 | 21 | 15 | 10 | 6 | 0 |
| tumor(mm) | | | | | | |

According to the observation of animal experiments, congestion occurred in the affected area of the animal tumor after injection, then suppuration occurred, and the tumor tissue eventually fell off the animal or was eliminated. This was because that this drug composition stimulated the recognition and killing activity of the immune system of the diseased animal to tumor cells, such that the malignant tumor was inhibited, and then the tumor was reduced or even eliminated.

These embodiments are only an explanation of this application, and do not limit the protection scope of this application. Those skilled in the art can make modifications without creative contribution to this embodiment after reading this specification, and it is protected by the patent law as long as it is within the scope of the claims of this application.

What is claimed is:

1. An anti-tumor drug composition, comprising an IL12 protein, a GMCSF protein, and an IL2 protein.

2. The anti-tumor drug composition according to claim 1, wherein in the composition, a mass ratio of the IL12 protein: the GMCSF protein: the IL2 protein is in ranges of 0.1-10:0.1-10:0.1-10.

3. The anti-tumor drug composition according to claim 2, wherein in the composition, the mass ratio of the IL12 protein: the GMCSF protein: the IL2 protein is in ranges of 0.6-6:0.6-6:0.6-6.

4. The anti-tumor drug composition according to claim 3, wherein in the composition, the mass ratio of the IL12 protein: the GMCSF protein: the IL2 protein is 1:6:2.

5. A polynucleotide composition, wherein the polynucleotide composition comprises one of the following:
(a) a polynucleotide composition coding the IL12 protein, the GMCSF protein, and the IL2 protein in claim 1; and
(b) a polynucleotide composition complementary to the polynucleotide composition in (a).


SEQUENCE LISTING (8 sequences)

SEQ ID NO: 1 - protein, 197 aa, Canis familiaris
RSLPTASPSPGIFQCLNHSQNLLRAVSNTLQKARQTLELYSCTSEEIDHEDITKDKTSTVEACLPLELTMNESCLASREISLITNGSCLASGKASFMTVLCLSSIYEDLKMYQMEFKAMNAKLLMDPKRQIFLDQNMLTAIDELLQALNFNSVTVPQKSSLEEPDFYKTKIKLCILLHAFRIRAVTIDRMMSYLNSS

SEQ ID NO: 2 - protein, 307 aa, Canis familiaris
IWELEKDVYVVELDWHPDAPGEMVVLTCHTPEEDDITWTSAQSSEVLGSGKTLTIQVKEFGDAGQYTCHKGGKVLSRSLLLIHKKEDGIWSTDILKEQKESKNKIFLKCEAKNYSGRFTCWWLTAISTDLKFSVKSSRGFSDPQGVTCGAVTLSAERVRVDNRDYKKYTVECQEGSACPSAEESLPIEVVVDAIHKLKYENYTSSFFIRDIIKPDPPTNLQLKPLKNSRHVEVSWEYPDTWSTPHSYFSLTFCVQAQGKNNREKKDRLCVDKTSAKVVCHKDAKIRVQARDRYYSSSWSDWASVSCS

SEQ ID NO: 3 - protein, 127 aa, Canis familiaris
APTRSPTLVTRPSQHVDAIQEALSLLNNSNDVTAVMNKAVKVVSEVFDPEGPTCLETRLQLYKEGLQGSLTSLKNPLTMMANHYKQHCPPTPESPCATQNINFKSFKENLKDFLFNIPFDCWKPVKK

SEQ ID NO: 4 - protein, 135 aa, Canis familiaris
APITSSSTKETEQQMEQLLLDLQLLLNGVNNYENPQLSRMLTFKFYTPKKATEFTHLQCLAEELKNLEEVLGLPQSKNVHLTDTKELISNMNVTLLKLKGSETSYNCEYDDETATITEFLNKWITFCQSIFSTLT

SEQ ID NO: 5 - protein, 197 aa, Felis catus
RNLPTPTPSPGMFQCLNHSQTLLRAISNTLQKARQTLEFYPCTSEEIDHEDITKDKTSTVEACLPLELTMNESCLASREISLITNGSCLASRKTSFMTTLCLSSIYEDLKMYQVEFKAMNAKLLMDPKRQIFLDQNMLTAIDELLQALNVNSVTVPQNSSLEEPDFYKTKIKLCILLHAFRIRAVTINRMMSYLNSS

SEQ ID NO: 6 - protein, 307 aa, Felis catus
IWELEKNVYVVELDWHPDAPGEMVVLTCNTPEEDDITWTSDQSSEVLGSGKTLTIQVKEFADAGQYTCHKGGEVLSHSFLLIHKKEDGIWSTDILREQKESKNKIFLKCEAKNYSGRFTCWWLTAISTDLKFTVKSSRGSSDPQEVTCGAATLSAEKVRVDNRDYKKYTVECQEGSACPAAEESLPIEVVVDAIHKLKYENYTSSFFIRDIIKPDPPKNLQLKPLKNSRHVEVSWEYPDTWSTPHSYFSLTFGVQVQGKNNREKKDRLSVDKTSAKVVCHKDAKIRVQARDRYYSSSWSNWASVSCS

SEQ ID NO: 7 - protein, 127 aa, Felis catus
APTSSPSSVTRPWQHVDAMKEALSLLNNSSEITAVMNETVEVVSEMFDPEEPKCLQTHLKLYEQGLRGSLISLKEPLRMMANHYKQHCPLTPETPCETQTITFKNFKEKLKDFLFNNPFDCWGPDQK

SEQ ID NO: 8 - protein, 134 aa, Felis catus
APASSSTKETQQQLEQLLLDLRLLLNGVNNPENPKLSRMLTFKFYVPKKATELTHLQCLVEELKPLEEVLYLAQSKNFHLNHIKELMSNINVTVLKLKGSETRFTCNYDDETATIVEFLNKWITFCQSIFSTLT